(19) United States
(12) Patent Application Publication
(10) Pub. No.: US 2009/0017477 A1
(43) Pub. Date: Jan. 15, 2009

(54) METHOD FOR DETERMINATION OF CONCENTRATION, CHARGE OR UNIT SIZE OF A SUBSTANCE

(76) Inventors: Harri HARMA, Turku (FI); Tero Soukka, Turku (FI)

Correspondence Address: JAMES C. LYDON, 100 DAINGERFIELD ROAD, SUITE 100, ALEXANDRIA, VA, 22314, US

(21) Appl. No.: 12/164,188

(22) Filed: Jun. 30, 2008

Related U.S. Application Data
(63) Continuation-in-part of application No. PCT/FI2006/000427, filed on Dec. 29, 2006.
(60) Provisional application No. 60/754,613, filed on Dec. 30, 2005.

(30) Foreign Application Priority Data
Jul. 2, 2007 (FI) 20070517

(51) Int. Cl. G01N 33/53; G01N 33/543

(52) U.S. Cl. 435/7.72; 436/518; 436/501

(57) ABSTRACT

This invention relates to a method comprising the steps of a) contacting a sample containing a sample substance and a solid phase comprising a signal element, and optionally a substance containing a signal element, i.e. a signal element substance; wherein at least one of said sample substance, said solid phase, and said signal element substance comprises a signal element; wherein the surface of the solid phase contains no specific binding partners; and wherein the solid phase is capable of binding said sample substance nonspecifically, preferably through adsorption, to said solid phase, and b) detecting a signal change resulted from binding of said sample substance and/or signal element substance to said solid phase, and/or change in distance from said sample substance and/or said signal element substance to said solid phase.

This application is a continuation-in-part of International Application PCT/FI2006/000427, filed Dec. 29, 2006, which claims benefit under 35 U.S.C. §119 of U.S. provisional application No. 60/754,613, filed Dec. 30, 2005 and Finnish patent application 20070517, filed Jul. 2, 2007.

### FIELD OF THE INVENTION

This invention relates to a method determining concentration, charge or unit size of a substance.

### BACKGROUND OF THE INVENTION

The publications and other materials used herein to illuminate the background of the invention, and in particular, cases to provide additional details respecting the practice, are incorporated by reference.

Determination of concentration or substances has been a fundamental part of the development pathway in understanding the world the way it is today in a number of different application areas. Therefore, multiple methods for measuring concentration have been developed. One such method relies on absorptivity of the sample of interest. Photometric measurement of, for example, proteins is based on the previous knowledge of molar absorptivity (U.S. Pat. No. 6,174,729). The second group consists of technologies were colour is formed as a result of a chemical reaction. Such technologies are, for example, Bradford, Lowry, biureat and Nanoorange methods (Sapan C V et al., Biotech. Appl. Biochem., 1999, 29, 99; Jones L J et al., Proteomic Technologies, 2003, 34, 850; Marshall T. Williams K M, Clin. Chem., 2000, 46, 392). Proteins have been precipitated using different strategies and compounds for the determination of protein concentration. Precipitants such as trichloroacetic acid, sulphosalicylic acid, benzethonium chloride and benzalkonium chloride have been applied to obtain aggregates which have been measured for concentration using nephelometry (Shephard M D, Whiting M J, Ann. Clin. Biochem., 1992, 29, 411). Also turbidimetry has been applied for the concentration measurement.

There are number of other technologies available for determination of concentration of the molecule of interest. These methodologies rely on the use of specific binding molecules. For example, fluorescence resonance energy transfer between soluble donor and acceptor units is typically based on specific binders such as antibodies and other ligands. The same applies to surface and particle-based resonance energy transfer methods (WO 98/15830; WO 00/23785; US 2004/0076948; FI 20030460; Kokko L et al., Anal. Chim. Acta, 2004, 503, 155). Other particle-based methods, such as luminescent oxygen channelling immunoassay and scintillation proximity assay, also utilize specific binders on particles to detect the presence of analyte molecules (U.S. Pat. No. 6,406,913; U.S. Pat. No. 6,524,786). Above-mentioned methods are separation-free system where no washing steps within the time course of the assay are performed. Well-known other separation-free assay methods are, for example, two-photon excitation, fluorescence polarization, fluorescence correlation, solid-surface scintillation and flow cytometric assays (Hanninen P et al., Nature Biotech. 2000, 18, 548; Park S H, Raines R T, Methods Mol. Biol. 2004, 261, 161; Thompson N L et al. Curr Opin Struct Biol., 2002, 12, 634; Earnshaw D L, Pope A J, J Biomol Screen., 2001, 6, 39; Fulton R J et al., Clin. Chem. 1997, 43, 1749).

A number of assays, which rely on the separation of bound and free labelled components in bioassays, are based mainly on enzyme and radioactive labels. Furthermore, fluorochromes have, traditionally, been used as detection dyes in assay concepts (Schonau A et al., J Immunol Methods, 1998, 218, 9; Soini E, Lovgren T. CRC Crit. Rev. Anal. Chem., 1987, 18, 105). Recently, dye particles have been applied to bioaffinity assays (Chan W C W, Nie S. Science, 1998, 281, 2016; Harma et al. Clin Chem, 2001, 47, 561). Again these methods are based on specific binding molecules to determine the sample of interest.

Unit size such as particle size of a sample has been traditionally measured using light scattering, photon correlation and polarization intensity differential scattering. These methods can typically measure size of a sample above 10 nm. Recently, back scattering method has been claimed to measure reliable sizes below 10 nm. Whether large or small particles are being measured, the existing methods rely largely on the use of scattering of the particulates.

### OBJECT AND SUMMARY OF THE INVENTION

One object of the present invention is to provide a method suitable for determining concentration, charge and/or unit size of a substance to be analyzed, i.e. a sample substance.

The present invention provides a method comprising the steps of

a) contacting a sample containing a sample substance and a solid phase comprising a signal element, and optionally a substance containing a signal element, i.e. a signal element substance; wherein at least one of said sample substance, said solid phase, and said signal element substance comprises a signal element; wherein the surface of the solid phase contains no specific binding partners; and wherein the solid phase is capable of binding said sample substance nonspecifically, preferably through adsorption, to said solid phase, and
b) detecting a signal change resulted from
i) binding of said sample substance and/or signal element substance to said solid phase, and/or
ii) change in distance from said sample substance and/or said signal element substance to said solid phase.

### BRIEF DESCRIPTION OF THE DRAWINGS

FIG. 1 illustrates the principle of a state-of-the-art method using specific binding partners on a solid surface. The surface contains a signal element and a specific binder is attached onto the surface. Fluorescence resonance energy signal is measured upon binding of a labelled competitive substance (A). When a sample substance is introduced the signal is altered and less resonance energy signal is detected (B).

FIG. 2 illustrates the principle of a surface recognition analysis. The signal of the solid-phase surface is altered upon binding of a sample substance.

FIG. 3 illustrates the principle of a competition analysis. A competitive substance (A) containing one member of a signal element pair binds onto the solid-phase containing the other member of a signal element pair and a signal is generated. Sample substance (B) is capable of binding onto solid-phase surface blocking the access of the competitive substance containing one member of a signal element pair to the solid phase.

FIG. 4 illustrates the principle of a complex analysis. First, sample substance, an antigen, is competing with a competitive substance, an antigen containing one member of a signal element pair, for a specific binding substance, an antibody (A). Thereafter, the complexes of sample and specific binding substances and/or competitive and specific binding substances are attached onto the solid-phase surface containing the other member of a signal element pair (B). The binding partners can also be added into the reaction volume simultaneously. The signal is altered and detected upon binding.

FIG. 5 illustrates the principle of a multi surface analysis. The sample substance is interacting with a solid surface (A). Thereafter or simultaneously, a particle containing a signal element is contacting the sample (B). The signal is monitored in a separation or separation-free assay format.

FIG. 6 illustrates a competition analysis and concentration measurement of bovine serum albumin on carboxylated europium(III)-chelate embedded nanoparticles. First, bovine serum albumin was attached to the surface of the nanoparticle and, thereafter, the remaining free surface sites were occupied by bovine serum albumin labelled with Alexa680 dye. The long-lived fluorescence energy transfer signal was monitored at 730 nm.

FIG. 7 illustrates a competition analysis of bovine serum albumin on Cy5-dyed surface. First, bovine serum albumin was attached onto the surface and, thereafter, the remaining free binding sites were occupied by europium(III)-labelled streptavidin. The time-resolved fluorescence signal was monitored at 665 nm. When the surface of the Cy5 was covered with albumin, no significant time-resolved fluorescence energy transfer signal was obtained.

FIG. 8 illustrates a competition analysis and concentration measurement of estradiol on carboxylated europium(III)-chelate embedded nanoparticles. First, estradiol was attached to the surface of the nanoparticle and, thereafter, the remaining free surface sites were occupied by estradiol labelled with Alexa680 dye. The long-lived fluorescence energy transfer signal was monitored at 730 nm.

FIG. 9 illustrates a competition analysis and concentration measurement of five different cell types on carboxylated europium(III)-chelate embedded nanoparticles. First, cells were attached to the surface of the nanoparticle and, thereafter, the remaining free surface sites were occupied by bovine serum albumin labelled with Alexa680 dye. The long-lived fluorescence energy transfer signal was monitored at 730 nm.

FIG. 10 illustrates a competition analysis and unit size measurement of monoclonal antibody or myoglobin proteins on carboxylated europium(III)-chelate embedded nanoparticles. First, monoclonal antibody or myoglobin was attached to the surface of the nanoparticle and, thereafter, the remaining free surface sites were occupied by antibody labelled with Alexa680 dye. The time-resolved fluorescence energy transfer signal was monitored at 730 nm. The competitive binding curve of the smaller myoglobin protein (molecular weight 17 000, diameter ˜3 nm) has shifted to higher concentration compared to the larger monoclonal antibody protein (molecular weight 160 000, diameter ˜10 nm) indicating size difference between the sample substances.

FIG. 11 illustrates a concentration measurement of bovine serum albumin on carboxylated microparticles using two-photon excitation technology. First, bovine serum albumin was attached to the surface of the microparticle and, thereafter, the BF530-dye embedded nanoparticles were adsorbed to the microparticle-bound bovine serum albumin. Two-photon excitation based TPX system was used to measure microparticle-bound BF530 nanoparticles at 560 nm.

FIG. 12 the principle of enzyme-based analysis. The solid phase contains an enzyme-specific substrate (A). The sample substance binds nonspecifically onto the solid-phase blocking the access of enzyme to its binding partner (B). If less sample substance is added a larger signal is monitored because more substrate is available for enzyme-mediated cleavage and signal generation.

FIG. 13 illustrates a competition analysis and concentration measurement of bovine serum albumin on 20, 40 or 80 nm gold nanoparticles. First, bovine serum albumin was attached to the surface of the nanoparticle and, thereafter, the remaining free surface sites were occupied by bovine serum albumin labelled with Eu(III) chelates. The long-lived fluorescence energy transfer signal was monitored at 614 nm. Left bar represents zero concentration and right bar bovine serum albumin at a 1.5 μM concentration.

FIG. 14 illustrates a competition analysis of bovine serum albumin on polystyrene nanoparticles incorporated with malachite green. First, bovine serum albumin was attached onto the surface and, thereafter, the remaining free binding sites were occupied by europium(III) chelate labelled antibody. The time-resolved fluorescence signal was monitored at 614 nm. When the surface of the nanoparticles was covered with albumin, a time-resolved fluorescence signal from non-bound Eu(III) chelate labelled antibody was obtained.

FIG. 15 illustrates a competition analysis and concentration measurement of HeLa cells on gold nanoparticles. First, cells were attached to the surface of the nanoparticle and, thereafter, the remaining free surface sites were occupied by bovine serum albumin labelled with Eu(III) chelate. The long-lived fluorescence energy transfer signal was monitored at 614 nm.

FIG. 16 illustrates a competition analysis and unit size measurement of sodium dodecyl sulfate on gold nanoparticles. First, sodium dodecyl sulfate was attached to the surface of the nanoparticle and, thereafter, Rhodamine B was allowed to interact with the nanoparticles. The fluorescence signal was monitored at 590 nm.

### DETAILED DESCRIPTION OF THE INVENTION

In one innovative aspect, the present invention relates to a method of determining concentration of a sample substance. In one embodiment, the invention relates to a method of determining unit size of a sample substance. Still according to one innovative aspect, the invention relates to a method of determining charge of a sample substance. Typical for the invention is that concentration, charge or size is being measured using a nonspecific interaction between solid-phase surface and sample substance or a complex containing sample substance as well as the proximity principle. The method can be used in measuring concentration, charge or size of sample substances in biological, organic or inorganic samples or in mixtures of such samples.

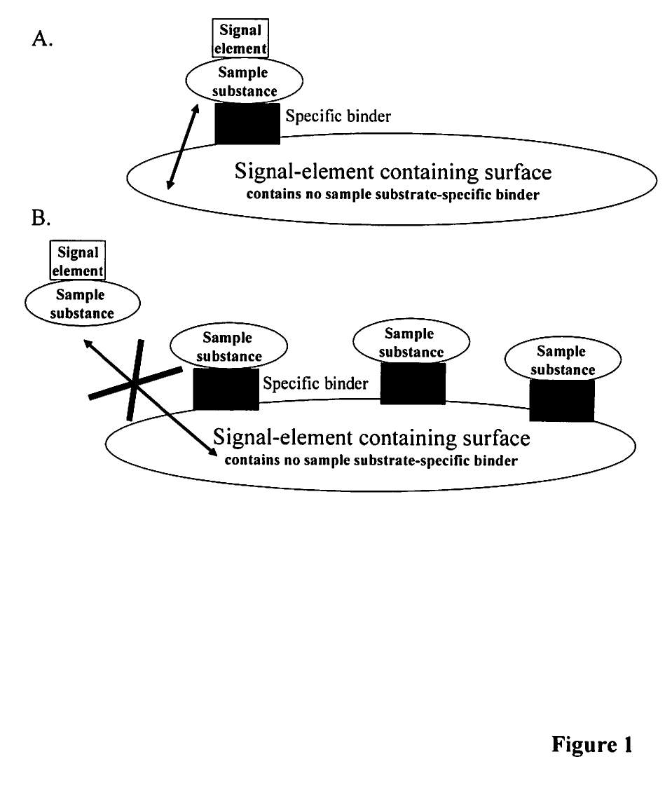

It is well known that proximity based assays have been performed in various biochemical bioaffinity assay set-ups. Characteristics for all these assays are that the assays utilize a specific binding partner (FIG. 1). It is also well known that nonspecific binding has been utilized to adsorb biomaterial onto various surfaces. It is also known that specific binding partners adsorbed onto labelled solid surface can be used to generate signal when a sample substance and a labelled ligand react specifically with the adsorbed binding partner in the reaction volume. However, prior to the present invention, it has not been known that using nonspecific adsorption of sample substance or a complex containing sample substance onto the solid surface can generate a significant signal, and this phenomenon is surprisingly applicable for determining concentration, size or charge of a sample substance. According to the invention a sample substance or a complex containing a sample substance binds directly onto the solid surface without any specific binders. High tendency or affinity towards the solid surface results in a high sensitivity analysis and low concentrations of sample substance can be measured.

The term “size” shall be understood as any measure of length, volume or repeating units of a sample substance. The term “solid-phase” or “surface” shall in this text be understood as any separable form of material. Thus it can be a particle or solid phase such as polymer, glass, or silica substrate. “Particle” means generally a particulate in the size region from 0.5 nm to 100 000 nm, preferably from 0.5 nm to 10 000 nm, more preferably from 0.5 nm to 1000 nm and most preferably from 0.5 nm to 500 nm. The particles may be unstable in solution. This means that particles may sedimentate within a relatively short period of time. Preferably, however, the particles remain in solution for at least 1 hour, more preferably 3 hours and even more preferably 6 hours and most preferably 12 hours. Alternatively, particle solution may be stable in solution. Preferably particle solution is colloidal, i.e. a stable dispersion of particles in solution. Particles can take any form, for example spherical, elliptic, rod or irregular shape. The particle can be formed from single entity, multiple entities, multiple different entities or an agglomerate of one or multiple entities. For example one can think of having a protein and agglomerate of proteins, or a small molecule such as a dye and agglomerate of dyes, or a polymer and agglomerate of polymers, or a mixture of proteins, dyes and polymers. The size distribution of the solid phase can be unimodal (monodisperse) or multimodal (polydisperse).

“Proximity” principle refers to the principle of a detection method where a signal is generated by close vicinity to a solid-phase and a substance containing a signal element or a sample substance comprising an inherent signal element. This may or may not mean that the sample substance is in contact with the surface. The signal may be generated by having a competitive substance in the detection volume. In such a case, the competitive substance carries a signal-generating unit and it competes for close vicinity to the solid-phase surface with the sample substance simultaneously or in sequential steps.

Nonspecific binding is determined as follows: The surface of the solid surface contains no specific binders to the sample substance. For example, a solid surface containing an antibody selectively binds to an antigen or a group of antigens with high affinity. Such a binding event is considered specific. Also structure-recognizing surfaces as achieved using molecular imprinting are considered specific in this context. Specific binders recognize their ligands, analogues of the ligands and structures very similar to the ligand with high affinity—typically higher than 10⁷ M⁻¹ (affinity constant). Specific binders have high selectivity towards their ligands, analogues of the ligands and structures very similar to the ligand Molecular recognition elements (i.e. “specific binders or binding partners”) that are coupled to a solid phase to enable “specific binding” of ligand molecules are known from prior art (J. Sep. Sci. 2006:29; 719, Nature Biotech. 2005:23; 1257). The following list gives examples of such specific binders:
nucleic acids,
antibodies (a protein),
artificial protein binders,
peptides (small part of a protein),
enzymes (a protein),
lectins (a protein),
receptors (a protein),
hormones (proteins or small organic molecules),
aptamers (a nucleic acid sequence),
small organic molecules (haptens),
metal chelates,
imprinted polymers,
enzyme inhibitors,
artificial nucleic acid mimics,
carbohydrates, polysaccharides,
enzyme substrates,
whole cells,
cell surface antigens, and
cell surface receptors.

When the surface contains no specific binders at all, or preferably none of the above-mentioned specific binders, it is according to the invention considered a nonspecific binding surface where the sample may be adsorbed or bound. The surface of the solid-phase may or may not contain one or multiple functional groups, typically selected from the group consisting of —COOH, —NH₂, —CHO, —OH, -maleimide, -succinimide, -epoxy and polymers (such as polyimide or grafted groups) whereto sample substance can be physically or chemically bonded or adsorbed. Typically the solid surface containing surface groups do not contain specific binders and accordingly surface groups do not specifically bind sample substances compared to essentially similar molecules.

The following methods are examples of proximity methods, which can be applied in the present invention and any of the fluorescence method described in Topics in Fluorescence Spectroscopy by J P. Lakowicz can be utilized according to the invention (Plenum Press or Springer, New York, published in multiple volumes starting from 1991):

The term “resonance energy transfer” relates to a method, where donor compound is in a close vicinity to acceptor compound. This generates an energy flow from the donor to the acceptor leading to a detection scheme where signal is monitored through the donor or acceptor. Such a method is well known for example in fluorescence resonance energy transfer system where donor dye can be down or up converting dye. The donor is excited and as a consequence of the proximity principle acceptor dye is excited by the donor compound and signal is detected at the emission wavelength of the acceptor compound. There are number of resonance energy transfer methods such as fluorescence, time-resolved fluorescence, bioluminescence and luminescence resonance energy transfer. The resonance energy transfer can be considered as signal generating method or a method where signal is quenched. In the case of quenching, any element can be used to quench signal such as a dye or metal. Typical metal chelators used in time-resolved fluorometry are 3-(2-thienoly)-1,1-trifluoroacetone, 3-benzoyl-1,1,1-trifluoroacetone, coproporphyrins, porphyrins, 3-naphthoyl-1,1,1-trifluoroacetone, 2,2-dimethyl-4-perfluorobutyoyl-3-butanone, 2,2′-dipyridyl, phenanthroline, salicylic acid, phenanthroline carboxylic acid, aminophenanthroline, diphenylphenantroline, dimethylphenanthroline, bipyridylcarboxylic acid, aza crown ethers, trioctylphosphine oxide, aza cryptands, dibenzoylmethane, dinaphtoylmethane, dibiphenoylmethane, benzoylacetonato, phenylazodibenzoylmethane, dithienylpropanedione, 4,4′-bis(N,N-dimethylamino)benzophenone, tris(6,6,7,7,8,8,8,-heptafluoro-2,2-dimethyloctane-3,5-dione, (alkyloxyphenyl)pyridine-2,6-dicarboxylic acid and their derivatives. (Selvin P. Nature Struc. Biol. 2000:7; 730, Forster T. Discuss. Faraday Soc. 1959:27; 7)

The term “luminescent oxygen channeling immunoassay” refers here to a method where singlet oxygen is being transferred from its host compound or matter to an acceptor compound or matter when singlet oxygen host and acceptor are in a close vicinity to one another. Typically a particle donor and acceptor are used in the method. (Ullman E. F. et al. Clin. Chem. 1996:42; 1518)

“Scintillation proximity assay” relates to any method where radioactive label is in close vicinity to matter, which is capable of transforming radioactive emitters to light or other form of detectable signal. The matter can be a particle or solid-phase. (Hart H. E. et al. Mol. Immunol. 1979:16; 265, Bosworth N et al. Nature 1989:341; 167)

“Two-photon excitation” is a method, which relies on a small detection volume. The small volume separates the detection volume from the surrounding medium allowing separation-free assay format. In the method, dyed substance is bound to a solid-phase particle on which the concentration of the sample substance of interest is detected. (Hanninen P. et al. Nature Biotech. 2000:18; 548)

“Coincidence assay” format is an assay concept where two differently dyed particles are coated with e.g. antibodies. As an analyte molecule couples two differently dyed particles together, the presence of the analyte is being measured by detecting the presence of both dyed particles in a small volume. For example, two-photon excitation can be used to reduce the detection volume and separate the two-particle complex from the surrounding medium without any physical separation. The method also allows a concept where only one dyed particle is being used. The other particle can be replaced with soluble dyed substance. (Heinze K. G. Biophys. J. 2002:83; 1671, Heinze K. G. Biophys. J. 2004:86; 506)

“Fluorescence polarization assay” refers to a method where polarization property of a dyed molecule is altered upon contact with another molecule. As the small dyed molecule is freely moving and rotating in medium, low polarization value is measured because movement and rotation occurs fast. As the small dyed molecule is attached to a larger molecule such as to a particle, its polarization is altered and a larger polarization value is measured. (Park S. H. et al. Methods Mol. Biol. 2004:261; 161)

“Fluorescence correlation spectroscopic assay” can be constructed using a particle and a dyed substance. As the dyed substance is being attached to the particle its fluorescence fluctuation pattern alters leading to a change in signal from freely fluctuating dyed substance. (Krichevsky O. at al. Rep. Prog. Phys. 2002:65; 251)

“Flow cytometric assay” is a separation-free assay format where particles are used as a solid-phase and dyed substance is attached on the surface of the particle. Thereafter, the extent of dyed substance is being detected through a flow cytometric system. The particles used are typically labelled with fluorochromes to identify each particle. (Fulton R. J. et al. Clin. Chem. 1997:43; 1749)

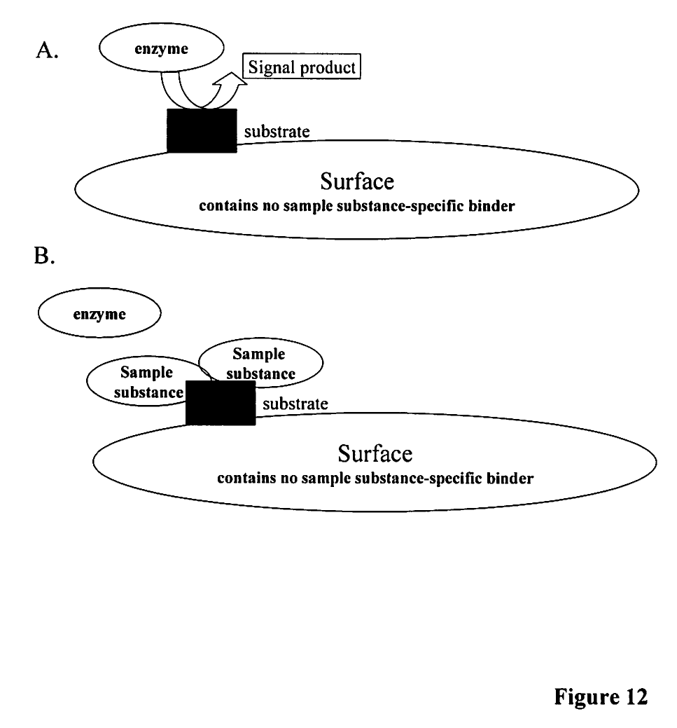

“Enzyme-based assay” relates to an assay format where substrate has been attached on a particle or solid-phase and soluble enzyme reacts with the solid-phase bound substrate molecule generating a detectable signal. As the substrate on a solid phase is typically a small compound, it can be blocked or coated with samples of interest blocking the enzyme activity. The change in the signal is evident as the enzyme is not capable of reacting with the substrate and generating signal (FIG. 12).

“Electron-based method” can be constructed using a group capable of releasing or receiving an electron. For example, substances labelled with ruthenium complexes are allowed to compete with a sample substance. As the labelled substance is in a close vicinity to an electrode, light is generated as the ruthenium complex undergoes a redox cycle. In another example, a lanthanide chelate labelled substance is allowed to compete with a sample substance. As the labelled substance is in a close vicinity to an electrode, light is generated. (Kenten J. H. Non-radioactive Labeling and Detection of Biomolecules. Springer Berlin, 1992; 175, Knight A. W. Trends Anal. Chem. 1999:18; 47)

“Nephelometry or turbidimetry” utilizes the enhanced scattering signal caused by particles. Typically, particles aggregate upon addition of sample substance. (Price C. P. at al. In Principle and practice of immunoassay. Macmillan Reference Ltd., London, UK, 1997; 579)

“Scattering material” refers to particles, for example gold or silver particles, and, when referring to surface-enhanced Raman scattering material, it can refer to, e.g. gold or silver particles coated with fluorescent molecules such as cyanine dye. Such scattering material can be used to recognize sample substance nonspecifically. (Ni J. et al. Anal. Chem. 1999:71; 4903, Schultz S. Proc. Natl. Acad. Sci. U.S.A. 2000:97; 996)

“Conducting metal particles” have typically resonance effects. These resonance effects can be utilized according to the invention to obtain a measurable signal. For example, the resonance effects of silver or gold nanoparticles can be used to enhance the fluorescence signal of fluorochromes close to the surface of the particles. A sample substance can affect this signal when nearing the surface or when adsorbed onto the surface. (Geddes C. D. et al. J. Fluor. 2002:12; 121)

Also magnetic properties of “magnetic solid surfaces” can be utilized according to the invention to generate a signal. For example, a sample substance is adsorbed on a solid surface non-specifically, and magnetic particles are used to bind nonspecifically to the sample substance, and the presence of the sample substance on the solid surface can be detected. (Baselt D. R. et al. Biosens. Bioelectron. 1998:13; 731)

According to the invention signal reduction can take place for example through quenching of a signal element or through adsorption of a signal element on a solid phase or through incorporation of a signal element into a solid phase. Quenching refers to a principle of a detection method where a signal is quenched by close vicinity of a substance containing a signal element or a sample substance comprising an inherent signal element to a solid-phase capable of quenching the signal of the substance containing a signal element or the sample substance comprising an inherent signal element. Quenching also refers to the principle of a detection method where a signal is quenched by close vicinity of a substance containing a signal reducing element or a sample substance comprising an inherent signal reducing element to a solid-phase comprising a signal element. This may or may not mean that the sample substance is in contact with the surface. The signal may be reduced by having a competitive substance in the detection volume. In such a case, the competitive substance carries or possesses a signal element and it competes from the vicinity to the solid-phase surface with the sample substance simultaneously or in sequential steps or the competitive substance carries or possesses a signal reducing element and it competes from the vicinity to the solid-phase surface comprising a signal element with the sample substance simultaneously or in sequential steps. Signal reduction may occur through e.g. excited state reactions, molecular rearrangements, energy transfer, complex formation, and static, dynamic or collisional quenching. Quenching is often heavily dependent on pressure and temperature. This can be applied to enhance signal reduction effects. Any of the signal reduction methods described in Principles of Fluorescence Spectroscopy (J. P. Lakowicz, Kluwer Academic/Plenum Publishers, New York, 1999) and Topics in Fluorescence Spectroscopy b (J. P. Lakowicz, Plenum Press or Springer, New York, published in multiple volumes starting from 1991) can be utilized according to the invention. Adsorption or incorporation of signal elements onto or into a solid phase may withdraw signal elements out of the focal area. This may take place simultaneously with signal reducing elements. In addition, solution transfer from one vial or reaction chamber or detection chamber to another may leave behind signal elements onto or into a solid phase reducing measured signal.

Properties of the surface of the solid-phase can be altered using multiple different compounds. The change in surface properties may lead to reduced or improved binding properties of the sample substance, to selective binding of different sample substance e.g. nucleic acids are bound but proteins are not bound or short polymers are bound but long polymers are not bound, to varied binding of the sample substance depending on test conditions, e.g. at low pH the sample substance is bound but at high pH the sample substance is not bound. Such compounds can be for example thiol amine (primary, secondary or tertiary), carboxy, aldehyde, ketone, hydroxyl, hydrophobic, hydrophilic, ionic, organic or inorganic groups facing one end of the compound and thiol amine (primary, secondary or tertiary), carboxy, aldehyde, ketone, hydroxyl, hydrophobic, hydrophilic, ionic, organic or inorganic groups facing the other end of the compound. The surface can be altered using one kind of compound or a combination of the compounds.

According to the invention, the change in signal can occur when the sample substance or substance containing a signal element is attached to or is nearing the solid-phase. The attachment can occur through physical or chemical bonding or adsorption. In addition, the change in signal can occur when the sample or substance containing a signal element is in a close proximity to the solid-phase without any physical or chemical bonding or adsorption. In the context of this invention adsorption refers to the phenomena wherein substances accumulate on a solid phase forming a molecular layer on the surface due to intermolecular attraction forces, i.e. van der Waals forces and not due to true chemical bonds, i.e. covalent bonds through electron share.

The methods listed above are based on the detection of luminescence such as fluorescence, phosphorescence, time-resolved fluorescence or up-converting fluorescence. The light may be generated using radioactivity, electrons, singlet oxygen or enzyme substrates. Also detection methods based on resonance effects, scattering or magnetic signal can utilized.

There are at least six preferable ways to perform an assay according to the present invention.

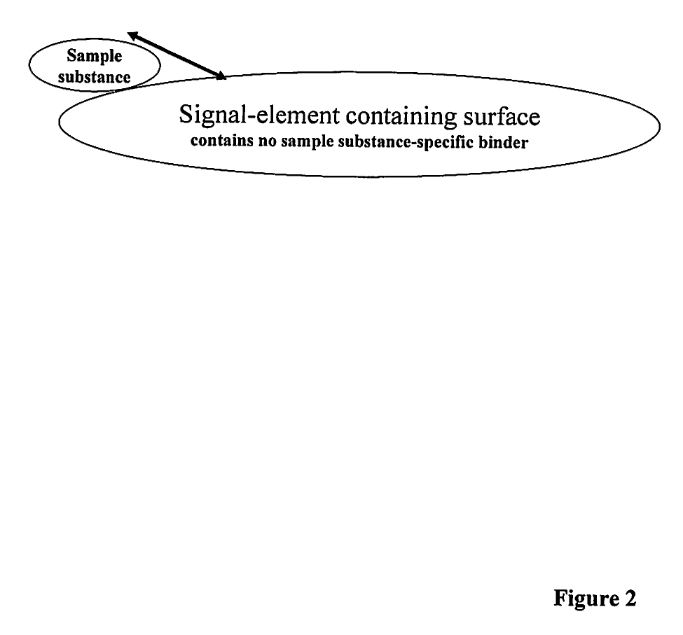

### 1. Surface Recognition Analysis (FIG. 2)

A signal element of the solid-phase is affected by the sample substance in a close vicinity to the solid-phase surface, to obtain a detectable change in the signal of the signal element of the solid-phase surface or the signal of an inherent signal element of the sample substance that may be affected by the proximity of the solid-phase surface. Alternatively, the sample substance can aggregate solid-phase particles changing the signal or scattering properties from those of non-aggregated particles. The sample substance is attached to the surface or nearing the surface nonspecifically.

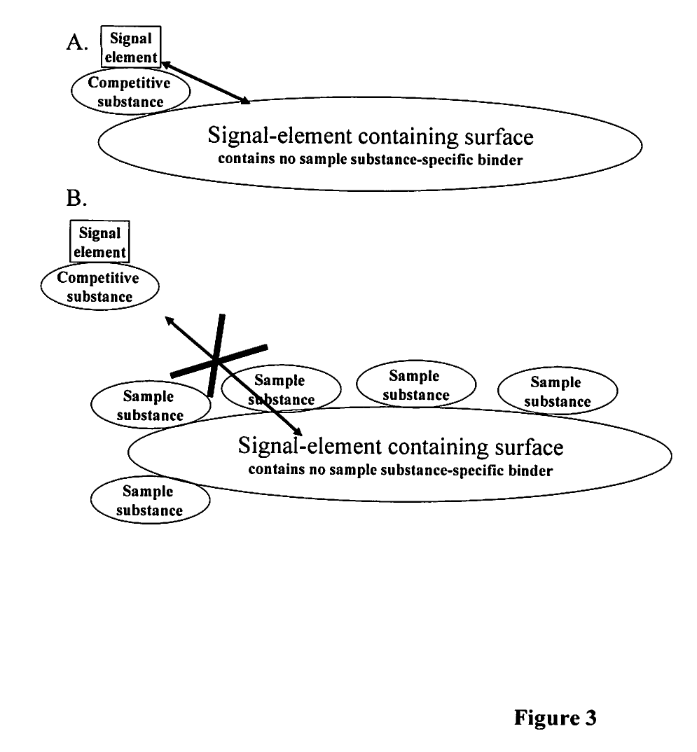

### 2. Competition Analysis (FIG. 3)

The sample substance can compete with a competitive substance for the proximity of the solid-phase surface. The competitive substance contains an element, which affects the signal of the solid-phase surface or the solid-phase surface contains an element, which affects the signal of the competitive substance. The competitive substance may have a high specificity and affinity toward the surface of the solid-phase surface. Typical is that the sample substance is capable of blocking the surface nonspecifically.

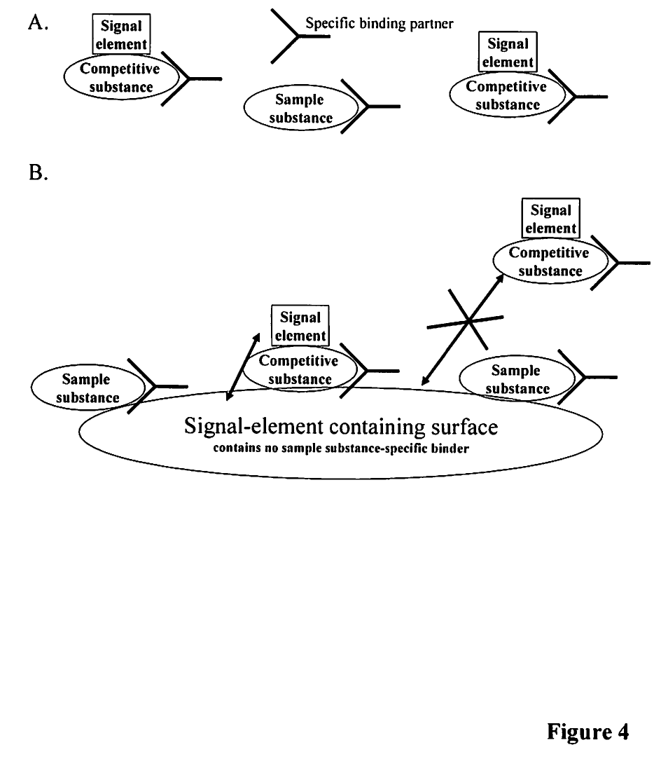

### 3. Complex Analysis (FIG. 4)

The sample substance is interacting with a second substance in a specific or nonspecific manner. This may occur competitively when using a competitive substance containing a signal element. The competitive substance may interact with the sample substance or the second substance. Thereafter the mixture of the complex, free sample substance, free substance containing a signal element and free second substance are brought in close vicinity to the solid-phase surface. The proximity of the mixture of the complex, the sample substance, the second substance and the competitive substance containing a signal element, in contact or near the solid-phase surface causes a detectable change in the solid-phase surface signal. The complex analysis can be used to detect a specific class of substances in the sample to be analyzed. The complex analysis according to the invention allows also interaction of, first, sample substance and, thereafter, the second labelled substance having an affinity toward the sample. This may also be performed in a non-sequential manner. The second substance may interact with the sample substance in a specific or nonspecific manner. The principles of the surface recognition and the competition analyses can be applied in the complex analysis. Typically no specific binders are used to couple the complex, free sample substance, free substance containing a signal element and/or free second substance onto the surface.

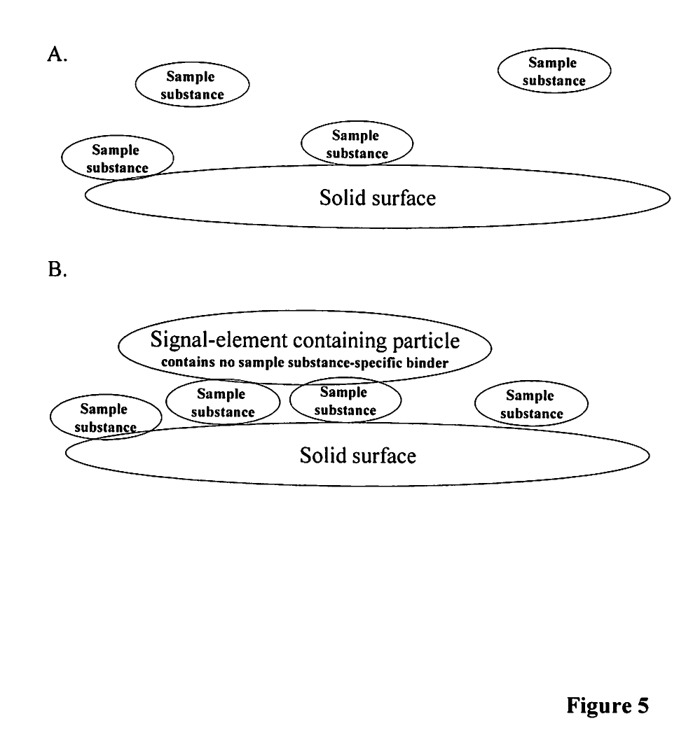

### 4. Multi Surface Analysis (FIG. 5).

Analyses can be performed using two or more solid surfaces. For example, a sample substance is in a close vicinity to a solid surface. The presence of the sample substance is recognized using a second solid surface for example a particle containing a signal element. The sample substance may be bound to the solid surface through specific or nonspecific interaction. Typical is that the particle containing the signal element has no specific interaction with the sample substance. According to one alternative, both solid surfaces may contain a signal element. The method can also be used by, first, allowing interaction of the sample substance and the particle, followed by adsorption onto the solid surface. The solid surface can be a solid material or a membrane containing pores or it can be made of nonporous material. The principles of surface recognition, competition and the complex analyses can be applied in multi surface analysis.

### 5. Focally Limited Analysis

A number of detection methods is based on focally limited volume in order to reduce background signal. This can be achieved for example using two-photon excitation, confocal or macroconfocal detection principles. In such methods the solid surface may or may not contain signal element. Therefore, the focal limitation analysis allows using a non-labelled solid surface. Detection technologies that can be used are for example cell counting by monitoring cells labelled nonspecifically with particles containing a signal element. The principles of surface recognition, competition, complex and the multi surface analyses can be applied in focally limited analysis.

### 6. Separation-Free Assay Format and Heterogeneous Assay Format

Surface recognition, competition, complex, multi surface, and focally limited analysis can be performed in a separation-free assay format without any need for a washing step. In addition, surface recognition, competition, complex, multi surface and focally limited analyses can be performed in a heterogeneous assay format where a washing step is required.

According to some preferred embodiments of the invention at least the solid phase comprises a signal element. According to other preferred embodiments of the invention at least the sample substance comprises an inherent signal element. Yet in other preferred embodiments of the invention the signal element substance is employed.

In some preferred embodiments the solid phase or surface is a particle.

In some preferred embodiments of the invention the signal element substance is a specific binding partner, which specifically binds with the sample substance, forming a complex which binds nonspecifically to the solid phase. In one embodiment of such embodiments the sample substance can bind to the solid phase and to a particle containing the signal element substance. In another embodiment of such embodiments the signal element substance competes with the sample substance in binding to the solid phase. In this embodiment a specific binding partner, which specifically binds with both the signal element substance and the sample substance, is contacted in step a) with the sample substance and the solid phase, and both the sample substance and the signal element substance forming complexes with the specific binding partner which complexes bind to said solid phase.

In some preferred embodiments the signal element substance is an enzyme substrate comprised in the solid phase; and an enzyme, capable of releasing a signal product from the signal element substance (if the signal element substance is not blocked by the sample substance bound or nearing the solid phase), is contacted in step a) with the sample substance and the solid phase.

In yet some preferred embodiments at least two different signal elements are employed and at least one signal element comprises one member of a label pair, and at least another signal element comprises the other member of the label pair, wherein the label pair s

i) a resonance energy transfer label pair
ii) a luminescent oxygen channeling immunoassay label pair, or
iii) a scintillation label pair.

In many preferred embodiments a detection principle involving focal limitation, preferably two-photon excitation, confocal or macroconfocal methods, is utilized.

The analyses of the methods of the invention can be performed in separation or separation-free format.

In preferred embodiments of the invention the signal change is used to measure the concentration of the sample substance, the charge of the sample substance and/or the unit size of the sample substance.

According to some preferred embodiments of the invention the solid phase comprises a signal reducing element. According to other preferred embodiments of the invention the sample substance comprises an inherent signal reducing element. Yet in other preferred embodiments of the invention a signal element substance comprising a signal reducing element is employed.

According to one alternative, the method of invention can be applied to measure a unit size of a sample substance. The unit size of a sample substance may vary from 0.1 nm to 100 000 nm in diameter. The unit size of a sample substance can be measured in solution or gas constituting of single sample substance or a mixture of sample substances. For example, the method can be used in detecting the size of a polypeptide. In the case of sample mixture, the method of invention can be used to measure unit size of a class of polypeptides. Particle surface may be constructed in a way that it discriminates between molecules of different sizes, such as DNA fragments of different length—large DNA is attached onto the solid-phase while short DNA is not. The surface is still not specifically constructed to bind DNA. The surface may well contain, for example, positive surface groups, which captures DNA but it also captures other polypeptides, for example, proteins.

The unit size can be measured, for example, using a known concentration of sample substance. According to the Stokes-Einstein relationship, diffusion is inversely related to the size of a substance. Therefore, a large sample substance has lower diffusion rate than a smaller sample substance. This rate can be utilized to measure the change in proximity signal and it can be related to the size of the sample substance. According to another strategy, the unit size of sample substance can be measured using the size-related change in proximity signal. Having the same concentration of samples substances, a large sample substance occupies a larger area on a solid-phase than a smaller sample substance. This may lead to a larger or different signal change in the case of larger sample substance. For example, a protein or aggregate form of the protein results in a different signal on a solid-phase due to different binding area.

According to still another strategy, the unit size can be measured using different binding properties of sample substance on a solid-phase. A large sample substance may bind on a solid-phase with a lower efficiency than a smaller sample substance or a smaller sample substance may bind on a solid-phase with a lower efficiency than a larger sample substance leading to a different change in proximity signal.

According to another embodiment, the method of invention can be used to determine charge of the sample substance. For example, in biochemistry, typically, protein pI value is measured and in polymer sciences, typically, particle surface charge is measured. The charge of sample substance can be measured using the size-related change in proximity signal. Having the same concentration of sample substances, a large sample substance may contain larger surface charge than a smaller sample substance. This may lead to a more efficient binding on a solid-phase and a larger signal change in the case of larger sample substance. For example, nucleic acids and aggregate or hybridized form of the nucleic acids results in different signal on a solid-phase due to different binding efficiency toward the surface of a solid-phase. According to another strategy, pH or salt concentration of a solution can be changed to bind differently charged substances onto solid phase. For example, two proteins with pI values of 5 and 8 can be tested by varying pH. When pH is chosen to be 3 both proteins carry positive charge and bind onto solid phase containing negative surface charge. Having pH 6 only the protein with pI value of 8 carries positive charge and binds onto the solid phase resulting in change in proximity signal.

“Sample” refers to a substance or substances to be analyzed, i.e. sample substance or substances, and optionally a carrier. Alternatively, the sample may contain the sample substance or substances only. The sample may contain solely a single substance. A sample substance can be any compound below 100 000 nm in diameter. The sample substance may appear in any form such as protein, nucleic acid, organic or inorganic substance, polymer, detergent, peptide, agglomerate, vesicle, liposome, particle or dyed particle as well as organism such as virus, bacterium or cell. Alternatively the sample substance may appear in mixtures of above-mentioned substances. Yet the sample substance may appear in a fragmented or disintegrated form where for example larger units, such as cells, are partially or totally broken into fragments or parts of said units. The sample substance can also be a biomolecule on a surface of any structure such as virus, bacterium, cell, vesicle, liposome, particle, polymer or any substance. The sample substance to be analyzed may inherently contain a signal element such as fluorescent or colour group, substrate for enzyme or the sample substance can be, for example, a fluorescent protein or particle.

The substance containing a signal element can be, for example, protein, nucleic acid, organic or inorganic substance, polymer, detergent, peptide, agglomerate, vesicle, liposome, particle or dye particle as well as organism such as virus, bacterium or cell. The signal element of the substance can be for example a luminophore, fluorescent protein, radioactive label, light producing or absorbing element, cleavable light producing or absorbing substrate for an enzyme, electron receiving or releasing material, scatterer, conducting metal, metal, carbon, inorganic or organic material, lanthanide metal dye, signal reducing element, semiconductor material, magnetic material or singlet oxygen producing element. The signal element may also be solid material containing a luminophore, fluorescent protein, radioactive label, light producing or absorbing groups, cleavable light producing or absorbing substrate for an enzyme, electron receiving or releasing solid material, scattering groups, conducting metal, metal, carbon, inorganic or organic material, lanthanide metal dye, signal reducing element, semiconductor material or singlet oxygen producing material. This is dependent on the method used. Any method can be used to attach the element to the competitive substance such as physical or chemical coupling.

The signal element of the solid-phase surface may be e.g. any element arising from the proximity methods or any of the following signal elements: luminescent, fluorescent, phosphorescent, light producing or absorbing element, cleavable light producing or absorbing substrate for an enzyme, time-resolved fluorescent or up-converting fluorescent element, metal, carbon, inorganic or organic material, lanthanide metal dye, signal reducing element, semiconductor material, magnetic material or singlet oxygen producing element. The signal may also be generated using radioactivity, enzymes, electrons or singlet oxygen. Also detection methods based on resonance effects, scattering or magnetic signal can utilized. A single signal element or multiple signal elements can be used. Cascades of different signal elements can also be utilized for example a combination of two or more dyes capable of undergoing resonance energy transfer between one another. The signal element may be inside the surface of the solid-phase. The signal element may be attached to the solid-phase by means of physical or chemical bonding or adsorption. In addition, the signal element may be coupled to a compound, which is attached onto or into the solid-phase. According to one alternative the solid phase may not contain any signal element.

The solid phase may be, for example, organic or inorganic material such as metal, semiconductor material or polymer. According to the invention also porous materials can be used. The size of a pore may be chosen to allow penetrating a reaction substance so that larger molecules cannot penetrate into the pores but smaller molecules are capable of penetrating. Essential is that no specific binding partners or specific binding pockets using imprinting technology is used to bind or allow the penetration the sample substance into the pores.

According to the invention kinetic and nonspecific binding properties can be aided using physical or chemical methods. For example, sonication can be conducted, pH, temperature or salt concentration can be varied or chaotropes can be added. Alternatively, by sonicating, by varying pH, temperature, salt concentration or by adding chaotropes breakage or denaturation of, for example, proteins or cells may occur leading to enhanced binding properties or increasing the number of detecting units. For example, breaking of a cell exposes cytoplasmic components into the reaction volume increasing the number of biomolecules in the reaction volume.

Typically any concentration or area of solid phase, any concentration of substance containing a signal element or sample can be used. However, in a competitive analysis it is preferred to use low concentration levels of the solid phase and the substance containing a signal element or a small area of the solid phase in order run sensitive assays. If solid phase particles are utilized it is preferable to work in a particle concentration range below 1 μM, more preferable <100 nM and most preferably <10 nM. In many preferred embodiments the particle concentration range is below 1 nM, preferably <100 μM, more preferably <10 μM and most preferably <1 μM. The concentration of the substance containing a signal element is preferable kept in a concentration range below 100 μM, more preferably <10 μM, and most preferably <1 μM. In many preferred embodiments the concentration of the substance containing a signal element is below 100 nM, preferably <10 nM, more preferably <1 nM, most preferably <100 μM. Because e.g. the substance containing a signal element and sample adsorb or interact with a large solid phase surface, the concentration of the solid phase is preferably lower than the concentration of the substance containing a signal element.

Typical signal reducing elements which can be used according to the invention may be luminophores or for example polymer, plastic, glass, silica, quartz, organic or inorganic material, metal, non-metals, carbon, semiconductor material or mixtures of these materials. Different metals can be applied for example gold, silver, copper, tin, nickel, aluminium, chromium, titanium, iron, platinum, zinc, cadmium, palladium, vanadium. Mixtures and alloys of metals such as steel, Anthracite iron, Cast iron, Pig iron, Wrought iron, Fernico, Elinvar, Invar, Kovar, Spiegeleisen or ferroalloys can be used. The signal reducing element can be chemical compound such as metal oxide, carbide, sulphite, phosphate, phosphide, nitride, fluoride, chloride, iodide or bromide. Various ions may act as signal reducers. Different carbon materials can be applied such as fullerenes, fullerenols, carbon black, carbon nanotubes, carbon nanorods, diamond, carbides, graphite.

According to the invention adsorption or interaction of sample substance, substance containing a signal element, second substance in a complex analysis or a complex in a complex analysis with a solid phase may occur through the sample substance, the substance containing a signal element, the second substance, the complex or through a signal element or a signal reducing element. Adsorption or interaction may also occur through two or more of the adsorbents or interactants.

According to the invention concentration of sample substance can be measured. In order to reduce the number of various components in the reaction volume, a separation step prior to the reaction may be required. This may well be the case in counting, for example, cells. Various separation methods can be, therefore, combined with the invention, for example, chemical or physical separation methods, centrifugation, aggregation, filtration or dialysis.

The analysis can be performed in an aqueous or organic phase or in a mixture of the two phases. The analysis can also be performed in an aqueous and/or organic phase in combination with a gaseous phase.

The method of invention can be used in multiple instrument setups. Whenever the sample substance must be excited with light, different configuration can be used to measure the concentration, charge or unit size of the substance successfully. The light source of such a measurement configuration can be, for example, halogen, xenon, tungsten, hydrogen or deuterium lamp or laser or semiconducting light source such as light emitting diode. The detector can be, for example, photoemissive, photomultiplier or semiconductor detector such as photodiode. The detecting configuration may occur by having the light source on one side of the measurement container and the detector on the other side. The light source and detector may well have an angle in between them. Often used setup is epiconfiguration (180 degree). Well-known methods such as filters, monochromators, prisms or gratings can be used to select suitable excitation and emission wavelength in case of optical configuration.

The method according to this invention can be used to measure concentration, charge or unit size of a sample substance in many various fields such as biology, biochemistry, chemistry, medicine, diagnostics, forensics, military, food industry, paper and pulp industry, paint industry, cosmetics.

The current invention provides several advantages. The invention provides very simple means for determining concentration, charge or unit size of a sample substance of interest. For example, typically a sample is mixed with solid-phase and optionally with a labelled competitive substance and the signal is monitored within a short period of time. The method does not require addition of further substances, change in temperature or pH, separation of bound and unbound components and long incubation times. No prior knowledge of light absorption properties is required. This is contrary to existing methods where molar absorptivity must be known, pH or temperature must be changed or long incubation times must be conducted for successful analysis. The method can be applied to many different areas of interest because both aqueous and organic solvents can be used to run an analysis. In addition, the method is highly sensitive. This stems from the fact that, for example, in the case of fluorescence resonance energy transfer the distance between donor and acceptor molecules may be very short. The distance is shorter than typically in a bioaffinity assay because the acceptor labelled substance is directly attached to the surface of the donor solid-phase surface and not through specific binding partner which increases the distance between the donor and the acceptor. This improves the sensitivity of the analysis as well as dynamic range of the analysis. In addition, high interaction tendency of sample substance with the solid phase improves the analysis performance. The sensitivity improvement can be a result of the reduced background due to the reduced number of proximity label pairs as more signal is available. Still another advantage can be found in favour of the invented method compared to the existing technologies for determination of concentration. Concentration of a fluorochrome-labelled sample is often difficult to measure accurately because fluorochromes may severely affect the spectral properties of the sample substance in spectrophotometric measurement. The fluorochrome is typically coupled to a protein through amine or thiol groups. For example, the Bradford method utilizes the very same groups in coupling the Bradford reagent to proteins to generated colour in solution. Therefore, it is difficult to standardize the method as similar protein with the same number of available functional groups should be found. The method of invention does not severely discriminate between substances and their dyed formats in attachment of the sample substance onto the solid-phase. The current invention also allows constructing inexpensive instrumentation.

Very few simple methods exist for measuring sub 10-nm size objects. Furthermore, the existing size determination methods are typically expensive highly dedicated instruments to measure scattering of objects under controlled conditions. The current invention offers a simple means to discriminate differently sized objects within minutes. Instrumentation is significantly less inexpensive compared to the light scattering methods.

The invention will be illuminated by the following non-restrictive Examples.

### EXAMPLES

### Example 1

### Competition Analysis and Determination of Protein Concentration

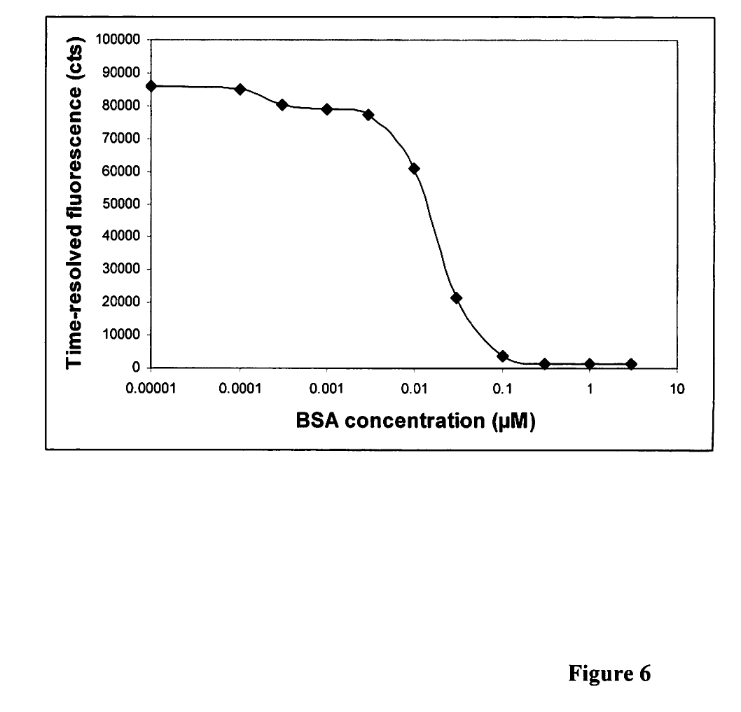

Carboxylated europium(III)-chelate embedded 107 nanometer particles were used as obtained from the manufacturer (Seradyn Inc, Indianapolis, Ind.). The nanoparticles were incubated with different concentrations of bovine serum albumin in 50 μL of 20 mM phosphate buffer, pH 7, for 10 min. Bovine serum albumin was adsorbed onto the nanoparticles. Thereafter, 10 ng of bovine serum albumin labelled with commercial N-hydroxysuccinimide Alexa680 dye (Molecular Probes, Eugene, Oreg.) was added to the sample-nanoparticle solution in 20 μL. The Alexa680-dyed bovine serum albumin occupied the remaining free sites on the nanoparticle. The solution was incubated for 10 min and measured for long-lived fluorescence signal at 730 nm using DELFIA 1234 time-resolved fluorescence plate reader (PerkinElmer Life and Analytical Sciences, Boston, Mass.). The excitation wavelength was 340 nm, the decay time of the measurement was 75 μs and the window time was 75 μs. The europium(III)-chelate nanoparticle acted as a donor and Alexa680 dye coupled to the bovine serum albumin served as an acceptor. The results are presented in FIG. 6.

### Example 2

### Competition Analysis and Determination of Protein Concentration

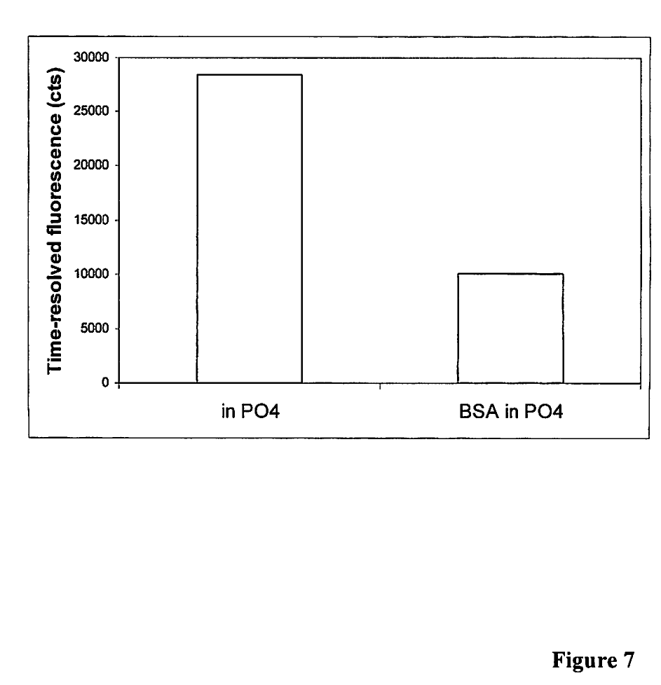

Cy5-labelled surface was prepared by coupling N-hydroxysuccinimide-Cy5 dye (Amersham, Buckinghamshire, UK) to an amino silane solid surface (silanized quartz). Phosphate buffer, 20 mM, pH 7, or phosphate buffer containing 100 ng of bovine serum albumin were incubated on the Cy5-surface in a 5 μL volume for 45 min. Both solutions contained 50 ng of europium(III)-labelled streptavidin as a competitive substance. The label used was 2,2′,2″,2′″-((2-(4-isothiocyanatophenyl)ethylimino)-bis(methylene)bis(4-((4-(α-galactopyranoxy)phenyl)ethynyl)-pyridine-6,2-diyl)bis(methylenenitrilo))-teterakis(acetato))europium(III). Europium(III)-labelled streptavidin was adsorbed onto the surface if no bovine serum albumin was in the solution. The europium(III)-labelled streptavidin acted as a donor and Cy5 dye coupled to the surface served as an acceptor. Upon binding onto the Cy5-labelled surface europium(III)-labelled streptavidin generated long-lived fluorescence signal at 665 nm. Long-lived fluorescence energy transfer signal was monitored using Victor 1420 time-resolved fluorescence plate reader (PerkinElmer Life and Analytical Sciences). The excitation wavelength was 340 nm, the decay time of the measurement was 75 μs and the window time was 100 μs. The results are presented in FIG. 7.

### Example 3

### Competition Analysis and Determination of Protein Concentration

A silver surface was prepared through physical vapour deposition technique. Phosphate buffer, 20 mM, pH 7, or phosphate buffer containing 100 ng of bovine serum albumin were incubated on the silver surface in a 5 μL volume for 45 min. Both solutions contained 50 ng of europium(III)-labelled streptavidin as a competitive substance. The label used was 2,2′,2″,2′″-((2-(4-isothiocyanatophenyl)ethylimino)-bis(methylene)bis(4-((4-(α-galactopyranoxy)phenyl)ethynyl)-pyridine-6,2-diyl)bis(methylenenitrilo))teterakis(acetato))europium(III). Europium(III)-labelled streptavidin was adsorbed onto the surface if no bovine serum albumin was in the solution. The europium(III)-labelled streptavidin acted as a donor and metallic silver on the surface served as a quencher. The long-lived fluorescence energy transfer signal was monitored at 615 nm using Victor 1420 time-resolved fluorescence plate reader. The excitation wavelength was 340 nm, the decay time of the measurement was 400 μs and the window time was 400 μs.

### Example 4

### Competition Analysis and Determination of Concentration of a Small Molecule

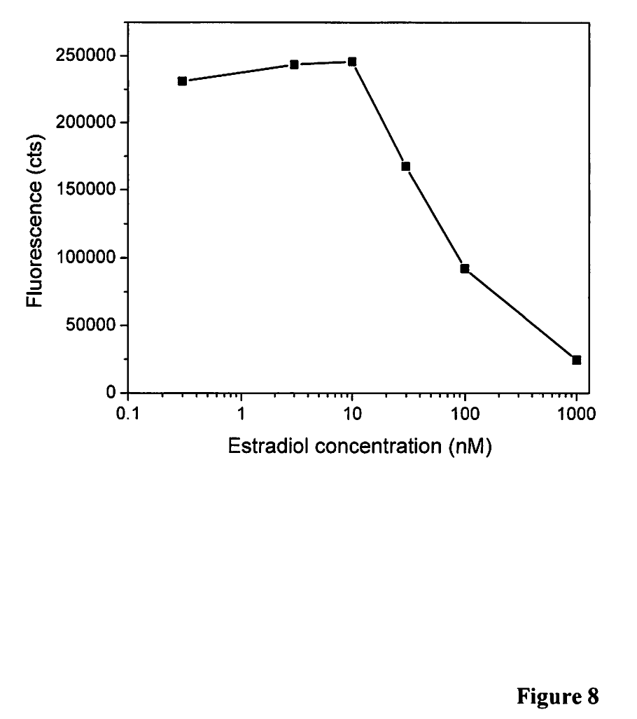

Carboxylated europium(III)-chelate embedded 107 nanometer particles were used as obtained from the manufacturer(Seradyn Inc, Indianapolis, Ind.). The nanoparticles were incubated with different concentrations of estradiol in 100 μL of 100 mM phosphate buffer, pH 7, for 10 min. Estradiol was adsorbed onto the nanoparticles. Thereafter, 10 nM solution of estradiol labelled with commercial N-hydroxysuccinimide Alexa680 dye (Molecular Probes, Eugene, Oreg.) was added to the sample-nanoparticle solution in 50 μL. The Alexa680-dyed estradiol occupied the remaining free sites on the nanoparticle. The solution was incubated for 10 min and measured for long-lived fluorescence signal at 730 nm using DELFIA 1234 time-resolved fluorescence plate reader (PerkinElmer Life and Analytical Sciences, Boston, Mass.). The excitation wavelength was 340 nm, the decay time of the measurement was 75 μs and the window time was 75 μs. The europium(III)-chelate nanoparticle acted as a donor and Alexa680 dye coupled to the estradiol served as an acceptor. The results are presented in FIG. 8.

### Example 5

### Competition Analysis and Determination of Cell Concentration

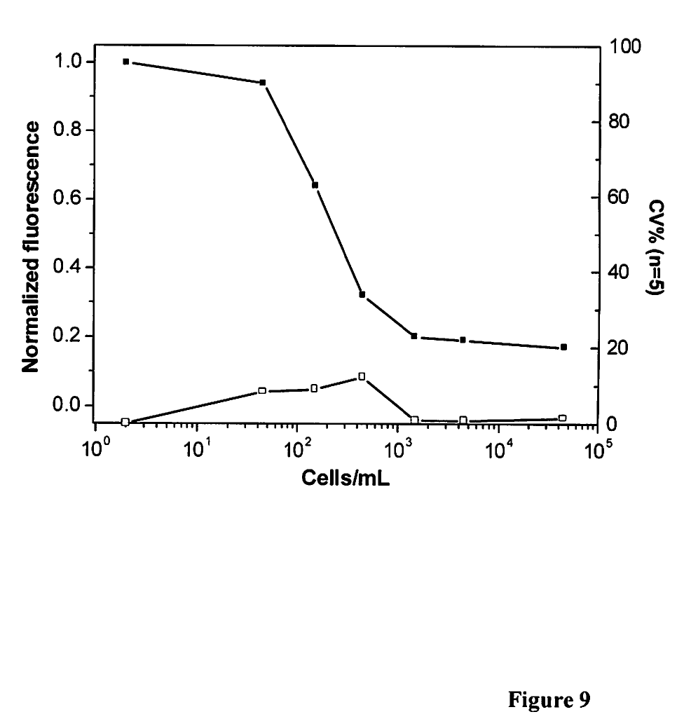

Carboxylated europium(III)-chelate embedded 107 nanometer particles were used as obtained from the manufacturer(Seradyn Inc, Indianapolis, Ind.). The nanoparticles were incubated with different concentrations of five different cell types in 50 μL of 20 mM phosphate buffer, pH 7, for 10 min. Cells were adsorbed onto the nanoparticles. Thereafter, 10 ng of bovine serum albumin labelled with commercial N-hydroxysuccinimide Alexa680 dye (Molecular Probes, Eugene, Oreg.) was added to the cell-nanoparticle solution in 20 μL. The Alexa680-dyed bovine serum albumin occupied the remaining free sites on the nanoparticle. The solution was incubated for 10 min and measured for long-lived fluorescence signal at 730 nm using DELFIA 1234 time-resolved fluorescence plate reader (PerkinElmer Life and Analytical Sciences, Boston, Mass.). The excitation wavelength was 340 nm, the decay time of the measurement was 75 μs and the window time was 75 μs. The europium(III)-chelate nanoparticle acted as a donor and Alexa680 dye coupled to the bovine serum albumin served as an acceptor. The results are presented in FIG. 9.

### Example 6

### Size Analysis of Proteins

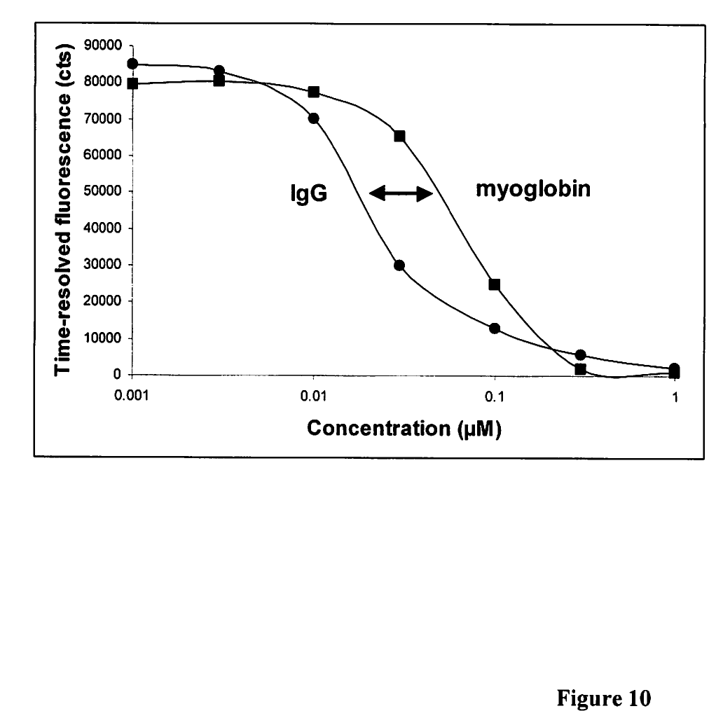

To elaborate the usefulness of the invented method for determining different sizes of sample substances, carboxylated europium(III)-chelate embedded 107 nanometer particles were utilized. First, the nanoparticles were incubated with different concentrations of monoclonal antibody or myoglobin in 50 μL of 20 mM phosphate buffer, pH 7, for 10 min. The proteins were adsorbed onto the nanoparticles. Monoclonal antibody and myoglobin have a molecular weight and size of 160 000, diameter ˜10 nm and 17 000, diameter ˜3 nm, respectively. Thereafter, 10 ng of antibody Fab fragment labelled with N-hydroxysuccinimide Alexa680 dye was added to the sample-nanoparticle solution in 20 μL. The Alexa680-dyed Fab fragment occupied the remaining free sites on the nanoparticle. The solution was incubated for 10 min and measured for long-lived fluorescence signal at 730 nm using DELFIA 1234 time-resolved fluorescence plate reader. The excitation wavelength was 340 nm, the decay time of the measurement was 75 μs and the window time was 75 μs. The europium(III)-chelate nanoparticle acted as a donor and Alexa680 dye coupled to the Fab fragment served as an acceptor. The different size of the proteins resulted in significantly different binding curves. The results are presented in FIG. 10.

### Example 7

### Multisurface Analysis of Proteins

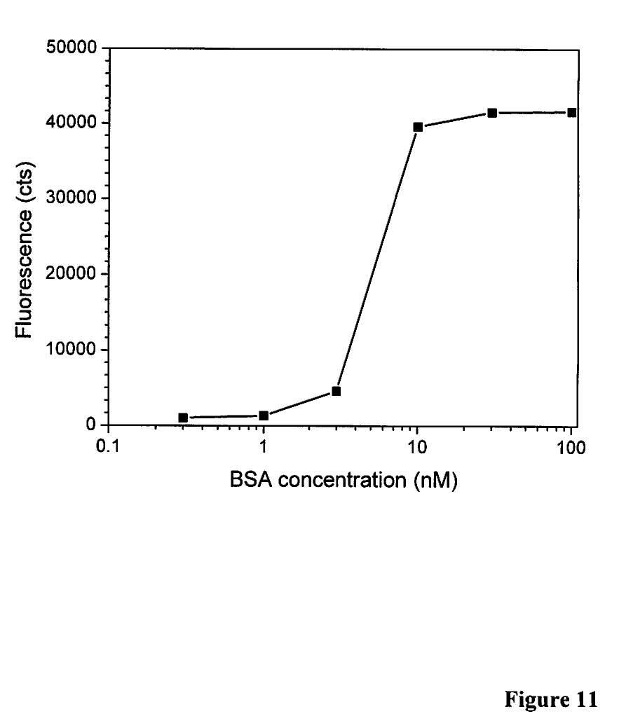

Carboxylated 3.2 micrometer particles were used as obtained from the manufacturer (Bangs Laboratories, Fishers, Ind.). The microparticles were incubated with different concentrations of bovine serum albumin in 15 μL of 20 mM phosphate buffer, pH 7, for 10 min. Bovine serum albumin was adsorbed onto the microparticles. Thereafter, 50 000 BF530-labelled nanoparticles of 53 nm in diameter (Arctic Diagnostics, Turku, Finland) was added to the sample-microparticle solution in 10 μL. The BF530-nanoparticles attached to the microparticle bound bovine serum albumin. The solution was incubated for 10 min and fluorescence was measured at 560 nm using two-photon excitation, TPX, system (Arctic Diagnostics, Turku, Finland). The excitation wavelength was 1064 nm. The focally limited detection technology measures microparticle whereto BF530-nanoparticle were bound through bovine serum albumin. The results are presented in FIG. 11.

### Example 8

### Competition Analysis and Determination of Protein Concentration

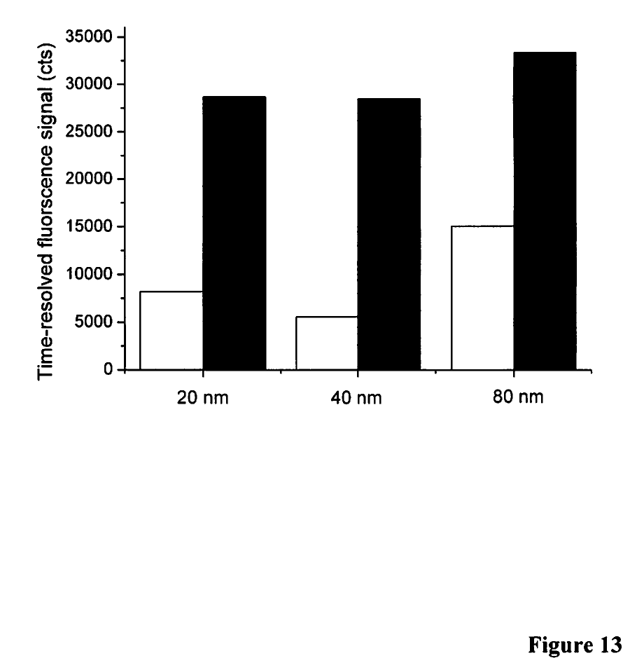

Gold nanoparticles of 20, 40 and 80 nm in diameter were used as obtained from the manufacturer (Brittish Biocell). The nanoparticles were incubated with 0 or 1.5 μM concentrations of bovine serum albumin in 50 μL of phosphate buffer (PBS), pH 7.4, for 10 min. Bovine serum albumin was adsorbed onto the nanoparticles. Thereafter, 1 ng of bovine serum albumin labelled with Eu(III) chelate (2,2′,2″,2′″-{[4-[(4-isothiocyanatophenyl)ethynyl]pyridine-2,6-diyl]bis(methylenenitrilo)} tetrakis (acetato)europium-(III), Eu(III) 7-dentate chelate) was added to the sample-nanoparticle solution in 20 μL. The solution was incubated for 30 min and measured for long-lived fluorescence signal at 614 nm using Victor 1420 time-resolved fluorescence plate reader (PerkinElmer Life and Analytical Sciences, Boston, Mass.). The excitation wavelength was 340 nm, the decay time of the measurement was 400 μs and the window time was 400 μs. The gold nanoparticles acted as a signal reducing element of Eu(III) chelate. The results are presented in FIG. 13.

### Example 9

### Competition Analysis and Determination of Protein Concentration

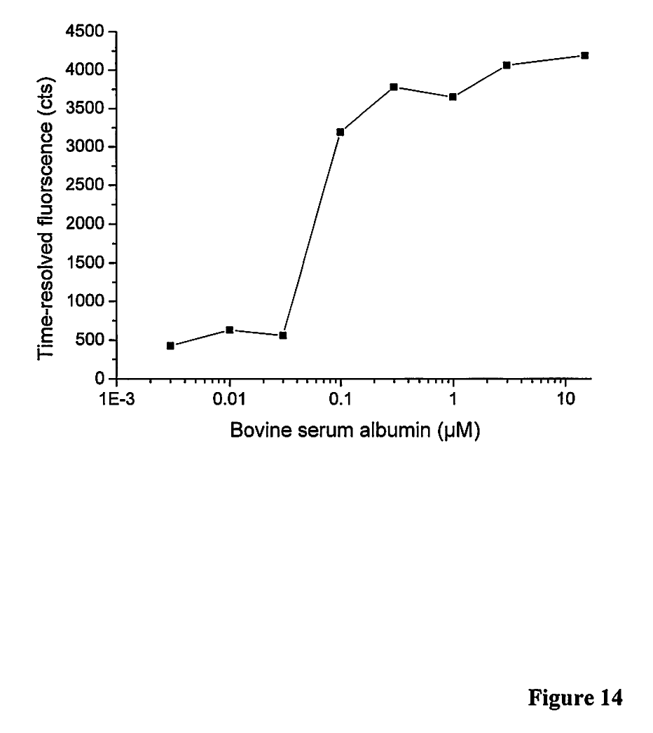

Polystyrene nanoparticles of 61 nm in diameter were prepared. Malachite green was incorporated into the prepared polystyrene nanoparticles. The nanoparticles were incubated in various concentrations of bovine serum albumin in 50 μL of phosphate buffer (PBS), pH 7.4, for 10 min. Bovine serum albumin was adsorbed onto the nanoparticles. Thereafter, 0.3 ng of antibody labelled with Eu(III) 9-dentate chelate ({2,2′,2″,2′″-{[2-(4-Isothiocyanatophenyl)ethylimino]bis(methylene)bis{4-{[4-(-galactopyranoxy)phenyl]ethynyl}pyridine-6,2-diyl}bis(methylene-nitrilo)}tetrakis(acetato)}europium(III)) was added to the sample-nanoparticle solution in 20 μL. The solution was incubated for 30 min and measured for long-lived fluorescence signal at 614 nm using Victor 1420 time-resolved fluorescence plate reader. The excitation wavelength was 340 nm, the decay time of the measurement was 400 μs and the window time was 400 μs. The polystyrene nanoparticles embedded with malachite green acted as a signal reducing element of Eu(III) chelate. The results are presented in FIG. 14.

### Example 10

### Competition Analysis and Determination of Concentration of Cells

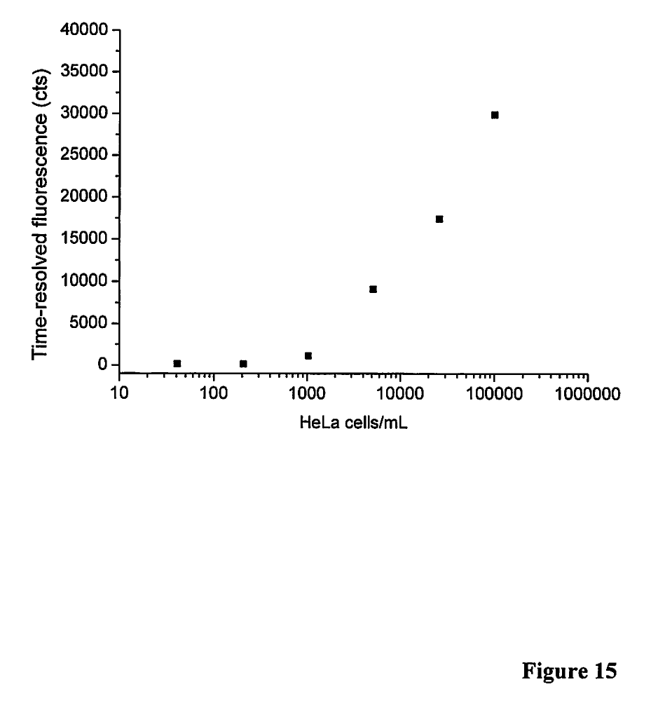

Gold nanoparticles of 40 nm in diameter were incubated with different number of cells in 50 μL of phosphate buffer (PBS), pH 7.4, for 10 min. HeLa cells were adsorbed onto the nanoparticles. Thereafter, 1 ng of bovine serum albumin labelled with Eu(III) 7-dentate chelate was added to the sample-nanoparticle solution in 12.5 μL. The solution was incubated for 40 min and measured for long-lived fluorescence signal at 614 nm using Victor 1420 time-resolved fluorescence plate reader. The excitation wavelength was 340 nm, the decay time of the measurement was 400 μs and the window time was 400 μs. The gold nanoparticles acted as a signal reducing element of Eu(III) chelate. The results are presented in FIG. 15.

### Example 11

### Competition Analysis and Determination of Various Compounds

Gold nanoparticles of 40 nm in diameter were incubated with different concentrations of PEG 6000, D-sorbitol, Dextran and polyvinylalcohol in 50 μL of phosphate buffer (PBS), pH 7.4, for 10 min. Sample substances were adsorbed onto the nanoparticles. Thereafter, 1 ng of bovine serum albumin labelled with Eu(III) 7-dentate chelate was added to the sample-nanoparticle solution in 25 μL. The solution was incubated for 30 min and measured for long-lived fluorescence signal at 614 nm using Victor 1420 time-resolved fluorescence plate reader. The excitation wavelength was 340 nm, the decay time of the measurement was 400 μs and the window time was 400 μs. The gold nanoparticles acted as a signal reducing element of Eu(III) chelate.

### Example 12

### Size Analysis of Polymers

Gold nanoparticles were incubated with 0 or 15 nM concentrations of linear polymer substance having molecular weight of 1000 or 15 000 Da in 50 μL of phosphate buffer (PBS), pH 7.4, for 10 min. Polymers were adsorbed onto the nanoparticles. Thereafter, 1 ng of bovine serum albumin labelled with Eu(III) 7-dentate chelate was added to the sample-nanoparticle solution in 15 μL. The solution was incubated for 30 min and measured for long-lived fluorescence signal at 614 nm using Victor 1420 time-resolved fluorescence plate reader. The excitation wavelength was 340 nm, the decay time of the measurement was 400 μs and the window time was 400 μs. The gold nanoparticles acted as a signal reducing element of Eu(III) chelate.

### Example 13

### Competition Analysis and Determination of Cells

Gold nanoparticles of 40 nm in diameter were incubated with different number of cells in 50 μL of phosphate buffer (PBS), pH 7.4, for 10 min. Bovine serum albumin was adsorbed onto the nanoparticles. Thereafter, Eu(III) chelate (2,2′,2″,2′″-[(4′-phenyl-2,2′,6′2″-terpyridine-6,6″-diyl)bis(methylenenitrilo)]tetrakis (acetic acid)) without a carrier was added to the sample-nanoparticle solution in 15 μL. The solution was incubated for 20 min and measured for long-lived fluorescence signal at 614 nm using Victor 1420 time-resolved fluorescence plate reader. The excitation wavelength was 340 nm, the decay time of the measurement was 400 μs and the window time was 400 μs. The gold nanoparticles acted as a signal reducing element of Eu(III) chelate.

### Example 14

### Competition Analysis and Determination of Bovine Serum Albumin

Carbon black nanoparticles were incubated with different concentration of bovine serum albumin in 50 μL of phosphate buffer (PBS), pH 7.4, for 10 min. Bovine serum albumin was adsorbed onto the nanoparticles. Thereafter, bovine serum albumin labelled with Eu(III) 7-dentate chelate was added to the sample-nanoparticle solution in 15 μL. The solution was incubated for 25 min and measured for long-lived fluorescence signal at 614 nm using Victor 1420 time-resolved fluorescence plate reader. The excitation wavelength was 340 nm, the decay time of the measurement was 400 μs and the window time was 400 μs. The carbon black nanoparticles acted as a signal reducing element of Eu(III) chelate.

### Example 15

### Competition Analysis and Determination of Sodium Dodecyl Sulfate

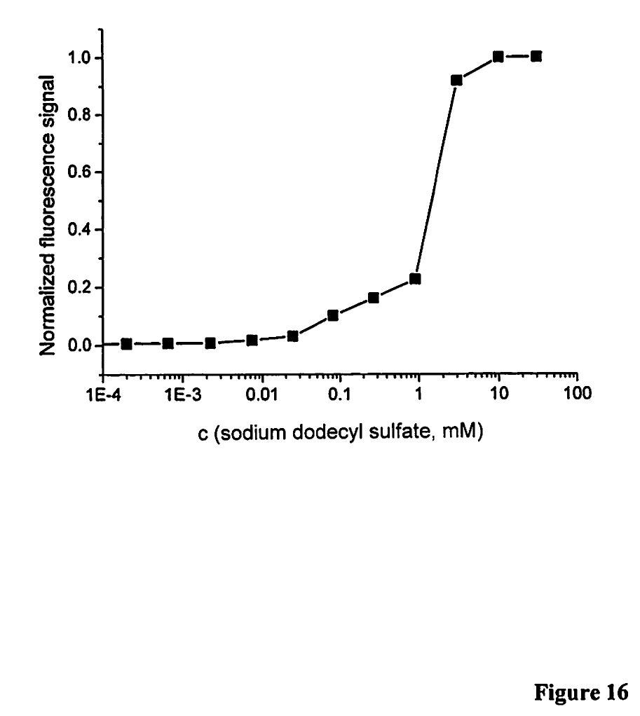

Gold nanoparticles of 40 nm in diameter were incubated with different concentration of sodium dodecyl sulfate in 50 μL of phosphate buffer (PBS), pH 7.4, for 10 min. Thereafter, Rhodamine B was added to the sample-nanoparticle solution in 15 μL. The solution was incubated for 20 min and measured for fluorescence signal at 485/590 nm using Tecan Ultra plate reader. This example shows how critical micelle concentration and surface tension can be measured using the method of invention. The results are presented in FIG. 16.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

1. A method suitable for determining concentration, charge and/or unit size of a substance to be analyzed, i.e. a sample substance, comprising the steps of
a) contacting a sample containing a sample substance and a particle comprising a signal element, and optionally a substance containing a signal element, i.e. a signal element substance; wherein at least one of said sample substance, said particle, and said signal element substance comprises a signal element; wherein the surface of the particle contains no specific binding partners; and wherein the particle is capable of binding said sample substance nonspecifically, preferably through adsorption, to said particle, and
b) detecting a signal change resulted from
i) binding of said sample substance and/or signal element substance to said particle, and/or
ii) change in distance from said sample substance and/or said signal element substance to said particle.

2. The method according to claim 1 characterized in that at least the sample substance comprises a signal element or a signal reducing element.

3. The method according to claim 1 characterized in that the signal element substance or a signal reducing element is employed.

4. The method according to claim 3 characterized in that the signal element substance is a specific binding partner, which specifically binds with the sample substance, forming a complex which binds nonspecifically to the particle and said signal element substance comprises a signal element or a signal reducing element.

5. The method according to claim 3 characterized in that the sample substance binds to a solid phase and to the particle containing the signal element substance.

6. The method according to claim 3 characterized in that the signal element substance competes with the sample substance in binding to the particle.

7. The method according to claim 6 characterized in that a specific binding partner, which specifically binds with both the signal element substance and the sample substance, is contacted in step a) with the sample substance and the particle, and both the sample substance and the signal element substance forming complexes with the specific binding partner which complexes bind to said particle.

8. The method according to claim 3 characterized in that the signal element substance is an enzyme substrate comprised in the particle; and an enzyme, capable of releasing a signal product from the signal element substance is contacted in step a) with the sample substance and the particle.

9. The method according to claim 1 wherein at least two different signal elements are employed characterized in that at least one signal element comprises one member of a label pair, and at least another signal element comprises the other member of the label pair, wherein the label pair is
i) a resonance energy transfer label pair
ii) a luminescent oxygen channeling immunoassay label pair, or
iii) a scintillation label pair.

10. The method according to claim 1 characterized in that a detection principle involving focal limitation, preferably two-photon excitation, confocal or macroconfocal methods, is utilized.

11. The method according to claim 1 characterized in that the analyses are performed in separation or separation-free format.

12. The method according to claim 1 characterized in that the signal change is used to measure the concentration of the sample substance.

13. The method according to claim 1 characterized in that the signal change is used to measure the charge of the sample substance.

14. The method according to claim 1 characterized in that the signal change is used to measure the unit size of the sample substance.
